(19) United States
(12) Patent Application Publication
(10) Pub. No.: US 2003/0211112 A1
(43) Pub. Date: Nov. 13, 2003

(54) EGFR ligands and methods of use

(76) Inventor: Waldemar Debinski, Hershey, PA (US)

Correspondence Address: Stanley A. Kim, Ph.D., Esq., Akerman Senterfitt, 222 Lakeview Avenue, Suite 400, West Palm Beach, FL, 33402-3188, US

(21) Appl. No.: 10/392,438

(22) Filed: Mar. 18, 2003

Related U.S. Application Data
(60) Provisional application No. 60/365,576, filed on Mar. 19, 2002.

(51) Int. Cl. A61K 39/395; C07H 21/04; C07K 16/46

(52) U.S. Cl. 424/178.1; 530/391.1; 536/23.53

(57) ABSTRACT

EGFR is over-expressed in malignant gliomas and, when activated, transduces apoptotic signals in these cancer cells. Chimeric molecules that contain an EGFR ligand and a carrier molecule are used to specifically target, and in some cases induce apoptosis in, EGFR-expressing cells.

### CROSS REFERENCE TO RELATED APPLICATION

The present application claims the benefit of U.S. provisional patent application No. 60/365,576 filed Mar. 19, 2002.

### FIELD OF THE INVENTION

The invention relates to the fields of medicine, immunology and oncology. More particularly, the invention relates to compositions and methods for killing cancer cells.

### BACKGROUND OF THE INVENTION

Epidermal growth factor (EGF) and transforming growth factor α (TGF-α) are cytokines that both interact with a cell surface receptor known as epidermal growth factor receptor (EGFR). EGFR is involved in the regulation of cellular differentiation and proliferation. EGFR activation results in a diverse array of signals that can result in changes in cellular proliferation, morphology, and differentiation. Production of TGFα by EGFR-expressing cells suggests that TGFα can act in an autocrine fashion to stimulate cell growth by constant activation of EGFR. EGFR is over-expressed in a large portion of human epithelial malignancies and malignant gliomas. Regarding the latter, approximately 50% of patients with glioblastoma multiforme (GBM) were observed to over-express the EGFR in situ. Activation of wild-type EGFR by increased levels of endogenous ligands, such as EGF, and use of a constitutively active EGFR, EGRFvIII, has indicated this receptor plays an important role in the etio-pathogenesis of malignant gliomas and their transformation into high-grade astrocytomas, such as GBM.

Several efforts to block EGFR function for the purpose of cancer treatment have emerged. Antibodies have been tested in an effort to restrict access of growth factors (e.g., EGF) to the receptor, thereby diminishing proliferative and oncogenic signals, and hampering tumor growth. Similarly, small molecules are being developed in an attempt to interfere with the signaling of activated EGFR.

The use of antibodies against EGFR is a very attractive approach for treating brain malignancies as normal brain and bone marrow express little if any EGFR. Several monoclonal antibodies (MAbs) have been raised against EGFR, of which 225, C225 and 425 are perhaps the most well characterized. MAb C225 induces apoptosis in cancer cells over-expressing EGFR, and MAb 425 has been tested in an early phase clinical trial in patients with malignant gliomas. In this clinical trial, significant anti-tumor and inflammatory responses were observed. These responses, however, were so dramatic that the associated edema forced the investigators to halt the trial. The trial did not show whether the anti-tumor response was mediated by (1) apoptosis triggered by engagement of the receptor, (2) classical antibody-mediated killing, or (3) a combination of both factors.

The use of different types of EGFR ligands should help elucidate the mechanism of this anti-tumor response. Such ligands should also be useful for treating tumors that overexpress EGFR.

### SUMMARY

The invention relates to the discovery that EGFR, when over-expressed in malignant gliomas, transduces apoptotic signals in these cancer cells. To specifically target EGFR, recombinant chimeric molecules have been developed that contain both an EGFR ligand and a carrier molecule. The EGFR ligand serves to direct the molecule to EGFR on a cell surface, while the carrier molecule serves to impart or alter a characteristic of the EGFR ligand. For example, the carrier molecule can (1) enhance the in vitro and/or in vivo stability of the chimeric molecule, (2) impart an effector function to the chimeric molecule, and/or (3) facilitate purification of the chimeric molecule. Useful examples of such chimeric molecules include TGF-α fused to a carrier molecule such as a mutated bacterial toxin or a portion of an immunoglobulin molecule. Contacting an EGFR-overexpressing cells such as a GBM cell with these chimeric molecules results in apoptotic cell death.

Accordingly, the invention features a chimeric molecule that include an epidermal growth factor receptor ligand and a carrier molecule. In one embodiment, the epidermal growth factor receptor ligand is TGFα. In some variations, the carrier molecule can be a portion of an immunoglobulin molecule such as one that includes CH2 and CH3 domains and a hinge region derived from an immunoglobulin. In other variations, the carrier molecule includes a bacterial toxin such as PE40Δ553/Δ609-613. Nucleic acid that encoding the foregoing chimeric molecules are also within the invention.

In another aspect, the invention features a method of inducing apoptosis in a cancer cell. This method includes the step of administering a composition including a chimeric molecule having an epidermal growth factor receptor ligand and a carrier molecule (e.g., one of the foregoing) in an amount effective to induce apoptosis in the cell. The cancer cell can be a glioma cell such as a glioblastoma multiforme cell.

Unless otherwise defined, all technical terms used herein have the same meaning as commonly understood by one of ordinary skill in the art to which this invention belongs. Commonly understood definitions of molecular biology terms can be found in Rieger et al., Glossary of Genetics: Classical and Molecular, 5th edition, Springer-Verlag: New York, 1991; and Lewin, Genes V, Oxford University Press: New York, 1994.

By the term “cancer” is meant any disorder of cell growth that results in invasion and destruction of surrounding healthy tissue by abnormal cells.

As used herein, “protein” or “polypeptide” means any peptide-linked chain of amino acids, regardless of length or post-translational modification, e.g., glycosylation or phosphorylation. The terms “chimeric molecule” and “chimeric protein” mean a protein molecule that consists of at least a first domain linked to a second domain in an arrangement that does not occur naturally.

By the term “ligand” is meant a molecule that will bind to a complementary site on a given structure. For example, an EGFR ligand binds EGFR.

When referring to a chimeric molecule, the term “carrier molecule” means any molecule that confers a functional attribute to the chimeric molecule.

By the terms “TGF-α protein” or simply “TGF-α” is meant a natural or any modified form of TGF-α (transforming growth factor-alpha) including a deletion, addition, substitution or other mutation in a naturally occurring TGF-α molecule.

The term “PE” as used herein means a natural or any modified form of PE (Pseudomonas exotoxin) including a deletion, addition, substitution or other mutation in a naturally occurring PE molecule.

The term “specifically binds”, as used herein, when referring to a polypeptide (including antibodies) or receptor, refers to a binding reaction which is determinative of the presence of the protein or polypeptide or receptor in a heterogeneous population of proteins and other biologics. Thus, under designated conditions (e.g. immunoassay conditions in the case of an antibody), the specified ligand or antibody binds to its particular “target” (e.g. an EGFR ligand specifically binds to an EGF receptor) and does not bind in a significant amount to other proteins present in the sample or to other proteins to which the ligand or antibody may come in contact in an organism. Generally, a first molecule that “specifically binds” a second molecule has a binding affinity greater than about 10⁵ (e.g., 10⁶, 10⁷, 10⁸, 10⁹, 10¹⁰, 10¹¹, and 10¹² or more) moles/liter.

Although methods and materials similar or equivalent to those described herein can be used in the practice or testing of the present invention, suitable methods and materials are described below. All publications, patent applications, patents, and other references mentioned herein are incorporated by reference in their entirety. In the case of conflict, the present specification, including definitions will control. In addition, the particular embodiments discussed below are illustrative only and not intended to be limiting.

### BRIEF DESCRIPTION OF THE DRAWINGS

The above and further advantages of this invention may be better understood by referring to the following description taken in conjunction with the accompanying drawings, in which:

FIG. 1 is a schematic illustration of native Pseudomonas exotoxin (PE), its derivative (PE40Δ553), and TGFα-PE chimeric molecules.

FIG. 2 is a schematic illustration of a recombinant TGFα-immunoglobulin constant region domain chimeric molecule.

### DETAILED DESCRIPTION

The invention provides methods and compositions for inducing cell death in tumor cells by targeting the cells with chimeric molecules that bind EGFR on the cells' surfaces. A number of different tumor cells can be killed by the methods and compositions described herein. Examples of such cells include those that over-express EGFR such as cells derived from epithelial tumors as well as those derived from gliomas (e.g., low grade or high grade astrocytomas including glioblastoma multiforme).

The below described preferred embodiments illustrate adaptations of these compositions and methods. Nonetheless, from the description of these embodiments, other aspects of the invention can be made and/or practiced based on the description provided below.

### Biological Methods

Methods involving conventional molecular biology techniques are described herein. Such techniques are generally known in the art and are described in detail in methodology treatises such as Molecular Cloning: A Laboratory Manual, 2nd ed., vol. 1-3, ed. Sambrook et al., Cold Spring Harbor Laboratory Press, Cold Spring Harbor, N.Y., 1989; and Current Protocols in Molecular Biology, ed. Ausubel et al., Greene Publishing and Wiley-Interscience, New York, 1992 (with periodic updates). Various techniques using polymerase chain reaction (PCR) are described, e.g., in Innis et al., PCR Protocols: A Guide to Methods and Applications, Academic Press: San Diego, 1990. PCR-primer pairs can be derived from known sequences by known techniques such as using computer programs intended for that purpose (e.g., Primer, Version 0.5, (1991, Whitehead Institute for Biomedical Research, Cambridge, Mass.). Methods for chemical synthesis of nucleic acids are discussed, for example, in Beaucage and Carruthers, Tetra. Letts. 22:1859-1862, 1981, and Matteucci et al., J. Am. Chem. Soc. 103:3185, 1981. Chemical synthesis of nucleic acids can be performed, for example, on commercial automated oligonucleotide synthesizers. Immunological methods (e.g., preparation of antigen-specific antibodies, immunoprecipitation, and immunoblotting) are described, e.g., in Current Protocols in Immunology, ed. Coligan et al., John Wiley & Sons, New York, 1991; and Methods of Immunological Analysis, ed. Masseyeff et al., John Wiley & Sons, New York, 1992. Conventional methods of gene transfer and gene therapy can also be adapted for use in the present invention. See, e.g., Gene Therapy: Principles and Applications, ed. T. Blackenstein, Springer Verlag, 1999; Gene Therapy Protocols (Methods in Molecular Medicine), ed. P. D. Robbins, Humana Press, 1997; and Retro-vectors for Human Gene Therapy, ed. C. P. Hodgson, Springer Verlag, 1996.

### Chimeric Molecules

The invention provides chimeric molecules that include both an EGFR ligand and a carrier molecule. The EGFR ligand is used to target the chimeric molecule to an EGFR on a cancer cell, while the carrier molecule confers a functional attribute to the chimeric molecule. For instance, the carrier molecule can function to increase stability of the chimeric molecule (e.g., for in vitro storage or in vivo delivery); to impart an effector function to the chimeric molecule (e.g., immune response-stimulating, cytotoxicity, etc.); or to facilitate purification of the chimeric molecule.

EGFR ligands useful in the invention include any molecule that can bind to EGFR. The molecule can be naturally occurring or artificially made. For example, naturally occurring EGFR ligands include TGF-α, EGF, EGF-like proteins, and other naturally occurring polypeptide chains known to bind EGFR. Examples of artificially created EGFR ligands include EGFR-binding antibodies (e.g., monoclonal antibody, polyclonal antibody, and antibody fragments) and engineered variants or mutants of naturally occurring EGFR ligands. The EGFR ligands useful in the invention include those that cause activation of EGFR and those that do not.

Carrier molecules of the present invention include those molecules that increase the stability of the chimeric molecule (e.g., for in vitro storage or in vivo delivery); introduce an effector function to the chimeric molecule (e.g., immune response-stimulating, cytotoxicity, etc.); or facilitate purification of the chimeric molecule. For increasing the stability of the chimeric molecule compared to the native ligand, the carrier can be a protein that has been shown to stabilize molecules similar to the EGFR ligand in an in vitro storage or in vivo delivery setting. For example, carrier molecules for increasing the stability of the chimeric molecule include PE, PE derivatives, and one or more constant heavy region domains from an immunoglobulin molecule (e.g., a CH₂—CH₃ fragment). Other carrier molecules that can be used to stabilize the chimeric molecule can be identified empirically. For instance, a molecule can be screened for suitability as a carrier molecule by conjugating the molecule to an EGFR ligand and testing the conjugated product in in Vitro or in vivo stability assays.

In some applications, carrier molecules within the invention can be used to introduce an effector function to the chimeric molecule. For introducing an effector function to the chimeric molecule, the carrier molecule can be a protein that has been shown to possess cytotoxic or immune response-stimulating properties. For instance, carrier molecules for introducing a cytotoxic function to the chimeric molecule include PE, PE derivatives, diptheria toxin, ricin, abrin, saporin, pokeweed viral protein, and constant region domains from an immunoglobulin molecule (e.g., for antibody directed cell-mediated cytotoxicity). Chimeric molecules that contain a cytotoxic carrier molecule can be used to selectively kill cells. Representative examples of such cytotoxic chimeric molecules include TGF-α fused to a mutant form of PE as well as TGF-α fused to constant region domains from an immunoglobulin molecule (e.g., CH₂—CH₃ fragment).

For introducing immune response-stimulating properties to a chimeric molecule, carrier molecules within the invention include any known to activate an immune system component. For example, antibodies and antibody fragments (e.g., CH₂—CH₃) can be used as a carrier molecule to engage Fe receptors or to activate complement components. A number of other immune system-activating molecules are known that might also be used as a carrier molecule, e.g., microbial superantigens, adjuvant components, lipopolysaccharide (LPS), and lectins with mitogenic activity. Other carrier molecules that can be used to introduce an effector function to the chimeric molecule can be identified using known methods. For instance, a molecule can be screened for suitability as a carrier molecule by fusing the molecule to an EGFR ligand and testing the chimeric molecule in in vitro or in vivo cell cytotoxicity and humoral response assays.

In other applications, carrier molecules within the invention facilitate purification of the chimeric molecule. Any molecule known to facilitate purification of a chimeric molecule can be used. Representative examples of such carrier molecules include antibody fragments and affinity tags (e.g., GST, HIS, FLAG, and HA). Chimeric molecules containing an affinity tag can be purified using immunoaffinity techniques (e.g., agarose affinity gels, glutathione-agarose beads, antibodies, and nickel column chromatography). Chimeric molecules that contain an immunoglobulin domain as a carrier molecule can be purified using immunoaffinity chromatography techniques known in the art (e.g., protein A or protein G chromatography).

Other carrier molecules within the invention that can be used to purify the chimeric molecule can be readily identified by testing the molecules in a functional assay. For instance, a molecule can be screened for suitability as a carrier molecule by fusing the molecule to an EGFR ligand and testing the fusion for purity and yield in an in vitro assay. The purity of recombinant proteins can be estimated by conventional techniques, for example, SDS-PAGE followed by the staining of gels with Coomassie-Blue.

A number of other carrier molecules can be used to impart an effector function to the chimeric molecule. These include other cytotoxins, drugs, detectable labels, targeting ligands, and delivery vehicles. Examples of these are described in U.S. Pat. No. 6,518,061 and U.S. published patent application No. 20020159972.

Carrier molecules can be conjugated (e.g., covalently bonded) to a EGFR ligand by any method known in the art for conjugating two such molecules together. For example, the EGFR ligand can be chemically derivatized with an carrier molecule either directly or using a linker (spacer). Several methods and reagents (e.g., cross-linkers) for mediating this conjugation are known. See, e.g., catalog of Pierce Chemical Company; and Means and Feeney, Chemical Modification of Proteins, Holden-Day Inc., San Francisco, Calif. 1971. Various procedures and linker molecules for attaching various compounds including radionuclide metal chelates, toxins, and drugs to proteins (e.g., to antibodies) are described, for example, in European Patent Application No. 188,256; U.S. Pat. Nos. 4,671,958; 4,659,839; 4,414,148; 4,699,784; 4,680,338; 4,569,789; and 4,589,071; and Borlinghaus et al. Cancer Res. 47: 4071-4075 (1987). In particular, production of various immunotoxins is well-known within the art and can be found, for example in “Monoclonal Antibody-Toxin Conjugates: Aiming the Magic Bullet,” Thorpe et al., Monoclonal Antibodies in Clinical Medicine, Academic Press, pp. 168-190 (1982); Waldmann (1991) Science, 252: 1657; and U.S. Pat. Nos. 4,545,985 and 4,894,443.

Where the carrier molecule is a polypeptide, the chimeric molecule including the EGFR ligand and the carrier molecule can be a fusion protein. Fusion proteins can be prepared using conventional techniques in molecular biology to join the two genes in frame into a single nucleic acid, and then expressing the nucleic acid in an appropriate host cell under conditions in which the fusion protein is produced.

A EGFR ligand may be conjugated to one or more carrier molecule(s) in various orientations. For example, the carrier molecule may be joined to either the amino or carboxy termini of an EGFR ligand. The EGFR may also be joined to an internal region of the carrier molecule, or conversely, the carrier molecule may be joined to an internal location of the EGFR ligand.

In some circumstances, it is desirable to free the carrier molecule from the EGFR ligand when the chimeric molecule has reached its target site. Therefore, chimeric conjugates comprising linkages that are cleavable in the vicinity of the target site may be used when the carrier molecule is to be released at the target site. Cleaving of the linkage to release the carrier molecule from the EGFR ligand may be prompted by enzymatic activity or conditions to which the conjugate is subjected either inside the target cell or in the vicinity of the target site. When the target site is a tumor, a linker which is cleavable under conditions present at the tumor site (e.g. when exposed to tumor-associated enzymes or acidic pH) may be used. A number of different cleavable linkers are known to those of skill in the art. See, e.g., U.S. Pat. Nos. 4,618,492; 4,542,225; and 4,625,014. The mechanisms for release of an agent from these linker groups include, for example, irradiation of a photolabile bond and acid-catalyzed hydrolysis. U.S. Pat. No. 4,671,958, for example, includes a description of immunoconjugates comprising linkers which are cleaved at the target site in vivo by the proteolytic enzymes of the patient's complement system. In view of the large number of methods that have been reported for attaching a variety of radiodiagnostic compounds, radiotherapeutic compounds, drugs, toxins, and other agents to antibodies one skilled in the art will be able to determine a suitable method for attaching a given carrier molecule to an EGFR ligand.

### Methods of Delivering an EGFR Ligand to a Cell

The invention also provides a method of delivering an EGFR ligand to a cell. This method is useful, among other things, for directing a chimeric molecule including an EGFR ligand and a carrier molecule to a cell so that the carrier molecule can exert its function. For example, an EGFR ligand conjugated to a cytotoxin can be delivered to a target cell to be killed by mixing a composition containing the chimeric molecule with the target cell expressing a receptor that binds the EGFR ligand. As another example, an EGFR ligand conjugated to a detectable label can be directed to a target cell to be labeled by mixing a composition containing the chimeric molecule with the target cell expressing a receptor that binds the EGFR ligand.

Chimeric molecules of the invention can be delivered to a cell by any known method. For example, a composition containing the chimeric molecule can be added to cells suspended in medium. Alternatively, a chimeric molecule can be administered to an animal (e.g., by a parenteral route) having a cell expressing a receptor that binds the EFGR ligand so that the chimeic molecule binds to the cell in situ. The chimeric molecules of this invention are particularly well suited as targeting moieties for binding tumor cells that overexpress EGFR, e.g., epithelial tumor cells and glioma cells. Thus, the methods of the invention can be used to target a carrier molecule to a variety of cancers.

### Administration of Compositions to Animals

For targeting an EGFR-expressing cell in situ, the compositions described above may be administered to animals including human beings in any suitable formulation. For example, compositions for targeting an EGFR-expressing cell may be formulated in pharmaceutically acceptable carriers or diluents such as physiological saline or a buffered salt solution. Suitable carriers and diluents can be selected on the basis of mode and route of administration and standard pharmaceutical practice. A description of exemplary pharmaceutically acceptable carriers and diluents, as well as pharmaceutical formulations, can be found in Remington's Pharmaceutical Sciences, a standard text in this field, and in USP/NF. Other substances may be added to the compositions to stabilize and/or preserve the compositions.

The compositions of the invention may be administered to animals by any conventional technique. The compositions may be administered directly to a target site by, for example, surgical delivery to an internal or external target site, or by catheter to a site accessible by a blood vessel. Other methods of delivery, e.g., liposomal delivery or diffusion from a device impregnated with the composition, are known in the art. The compositions may be administered in a single bolus, multiple injections, or by continuous infusion (e.g., intravenously). For parenteral administration, the compositions are preferably formulated in a sterilized pyrogen-free form.

Systemic (i.v.) with local interstitial drug delivery may be used according to the invention. The concept of convection-enhanced delivery is becoming more attractive as an effective route of drug delivery into the brain. Laske et al., Nature Medicine 3, 1362-1368 (1997). Consequently, local delivery is the preferred approach to be evaluated clinically, since it may achieve high concentrations directly within a tumor mass and its vicinity.

The compositions used in the invention may be precisely delivered into tumor sites, e.g., into gliomas, by using stereotactic microinjection techniques. For example, the mammalian subject can be placed within a stereotactic frame base that is MRI-compatible and then imaged using high resolution MRI to determine the three-dimensional positioning of the particular tumor being targeted. According to this technique, the MRI images are then transferred to a computer having the appropriate stereotactic software, and a number of images are used to determine a target site and trajectory for composition microinjection. Using such software, the trajectory is translated into three-dimensional coordinates appropriate for the stereotactic frame. For intracranial delivery, the skull will be exposed, burr holes will be drilled above the entry site, and the stereotactic apparatus positioned with the needle implanted at a predetermined depth.

### EXAMPLES

The present invention is further illustrated by the following specific examples. The examples are provided for illustration only and are not to be construed as limiting the scope or content of the invention in any way.

### EXAMPLE 1

### Fusion Proteins

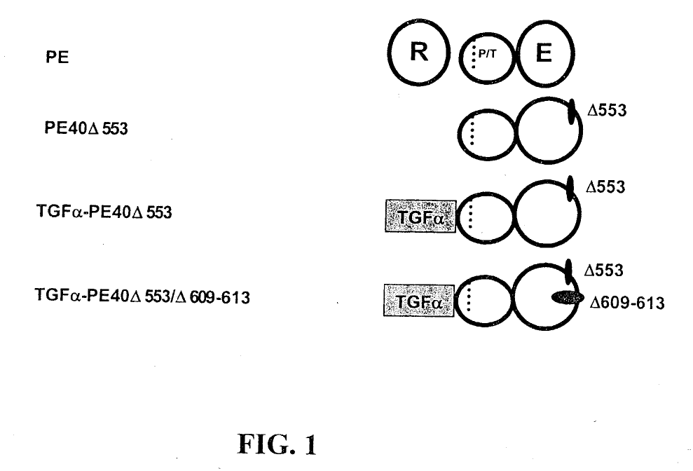

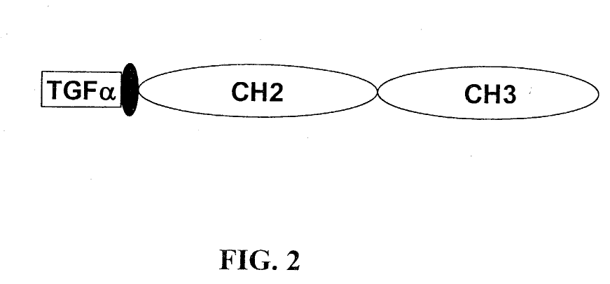

Referring to FIGS. 1 and 2, several EGFR-binding fusion proteins are illustrated. FIG. 1 shows two fusion proteins that include TGF-α as an EGFR-binding domain and a bacterial toxin (e.g., PE or mutants thereof). Both include TGF-α as the EGFR-ligand. TGF-α-PE40delta553 includes a mutant form of PE40 with a deletion at amino acid 553; whereas TGF-α-PE40delta553/delta609-613 includes a mutant form of PE40 with deletion at amino acid 553 and at amino acids 609-613.

FIG. 2 show a TGF-α-immunoglobulin fusion protein. Two different such proteins were made. In the first protein, TGF-α was fused to a human immunoglobulin G composed of the hinge region, constant heavy region 2 (CH₂) and CH₃ (FIG. 2). In the second protein, TGF-α was fused to a murine immunoglobulin G (isotype-matched to human Ig) composed of hinge region, CH₂ and CH₃ (TGF-α-mIG). These two proteins represent “immunoglobunoids” in which the antigen-binding region of the light and heavy chains of an Ig are replaced by TGF-α as an antigen (EGFR) binding domain (FIG. 2). The immunoglobunoids thus have the immunoglobulin functions, such as hinge regions and complement and macrophage binding, preserved (FIG. 2).

For preparing a TGF-α-IgG chimeric molecule, a plasmid encoding TGF-α and human IgG domains (i.e., hinge region, CH₂ and CH₃) is generated in a ligation process involving three DNA fragments. The first fragment, TGF-α, is amplified from the plasmid TGFα-PE40D553 by PCR to produce a fragment with NheI and BlpI cohesive ends. The second fragment, which is composed of a human immunoglobulin hinge region and second and third constant regions of the heavy chain, is also generated by PCR and contains BlpI and XbaI ends with a stop codon preceding the XbaI site. The third fragment is a commercially available vector (PVAX-1) that is digested within a polylinker region with NheI and XbaI restriction endonucleases. The large fragment liberated from the restriction digest is isolated and ligated to the first and second fragments. The ligation produces a gene that encodes a protein featuring TGF-A at the N-terminus and the CH₃ domain at the C-terminus. The plasmids encoding chimeric molecules are propagated and the proteins are expressed. Plasmids carrying the genes encoding proteins of interest are under a T7 promoter-based expression system as has been described previously for bacterial toxin expression. Debinski et al., Mol. Cell. Biol. 11:3:1751-1753, 1991; Debinski and Pastan, Cancer Res. 52: 53795385, 1992; and Debinski et al., J. Clin. Invest. 90:405-411, 1992.

To propagate these high-copy number plasmids, a high transformation efficiency strain of E. coli, such as HB11 or DH5α (Gibco-BRL), is used. BL21 (λDE3), which carries the T7 RNA polymerase gene in an isopropyl-1thio-β-galactopyranoside (IPTG) inducible form, is used as the host for recombinant protein expression. Proteins are purified using a Pharmacia fast protein liquid chromatography (FPLC) system. The purity of the recombinant proteins is estimated by SDS-PAGE followed by staining gels with Coomassie-Blue. Removal of endotoxins is performed by affinity chromatography (e.g., Detoxi-gel, Pierce Chemical).

### EXAMPLE 2

### Analysis of Purified Chimeric Molecules

Recombinant proteins are labeled with ¹²⁵I according to the standard lodo-Gen technique. Binding of the radiolabeled recombinant EGFR ligands is evaluated by Scatchard analysis on U-87 MG and U-251 MG glioma cells. The data is analyzed using the NIH Ligand program to determine K_{d} and Bₘₐₓ values, as described previously. Debinski et al., Clin. Cancer Res. 1: (Advances in Brief):1253-1258, 1995; Debinski et al., J. Biol. Chem. 271:22428-22433, 1996. In vitro stability of radiolabeled recombinant EGFR ligands is evaluated by adding equal aliquots (50-100 μl) to 2 solutions of phosphate-buffered saline (pH 7.4, 500 μl) and mixed on a rotator at 4° C. or at 37° C. A sample (20 μl) of each is removed at specific time intervals: 6, 12, 18, 24, 72, and 120 hrs, and screened by size exclusion HPLC.

### EXAMPLE 3

### Method of Testing EGFR Ligands for Ability to Induce Apoptosis in Cancer Cells In Vitro

The proliferative/anti-proliferative properties of EGFR ligands is monitored and an analysis of the indices of apoptosis is made in cells treated with various levels of EGF (1 to 50 ng/ml) or a corresponding amount of EGFR ligand for various periods of time (1 to 3 days). Cancer cells over-expressing EGFR are used in the assays. A few malignant glioma cells have been evaluated for this phenomenon including U-87 MG cells. These cells have been shown to succumb to apoptosis when 50 ng/ml instead of 20 ng/ml of EGF is added to the media. This result has been reproduced and a similar phenomenon has been observed in SN13-19 cells. Accordingly, these cells can be used in a method of testing EGFR ligands for the ability to induce apoptosis. In addition, other brain tumor cells such U-251 MG, T-89G, A-172 MG, and SF-295, which all are known to over-express the EGFR, as well as U-373 MG (relatively lower levels of the EGFR) and U-138 MG (low levels of EGFR) might be used. Non-brain tumor cells such as the commonly used epidermoid carcinoma A431 cells might also be used. These cells possess a large number of EGFRs and become apoptotic in the presence of a slightly elevated concentration of EGF. A431 cells are also tumorigenic, so can be used in comparative in vivo studies. Normal cells are used as a negative control.

Cell proliferative activity is tested using a colorimetric MTS [3-(4,5 -dimethylthiazol-2-yl)-5-(3 -carboxymethoxyphenyl)-2-(4-sulfophenyl)-2H-tetrazolium, inner salt]/PMS (phenazine methasulfate] cell proliferation assay, as described. Debinski et al., Clin. Cancer Res. 5:985-990, 1999. Clonogenic assays are performed using 5×10² of normal, e.g., HUVEC or malignant cells plated in triplicate gelatinized 100-mm Petri dishes. Recombinant EGF is added the following day at various concentrations for comparison with non-treated controls. The plates are incubated for a 10-14 day interval, the media is then removed and the colonies are fixed and stained with 0.25% crystal violet in 25% ethyl alcohol. The colonies containing greater than 50 cells are scored.

DNA fragmentation is analyzed as one of the measures of apoptosis. DNA is isolated from malignant and normal cells treated with various concentrations of EGF or EGFR ligands and electrophoresed on an agarose gel. Pulse field gel electrophoresis is utilized for quantitative analysis of double-stranded DNA fragmentation. The appearance of the cleavage fragments of poly(ADP)-ribosepolymerase (PARP) is determined to document the activation of caspase-3 which is the following step in the initiation of apoptosis by caspase-8 and -9 pathways.

### EXAMPLE 4

### Dead End Fluorometric Tunel System

Apoptosis in cell was measured using a dead end flurometric Tunel system as follows. Cells were plated onto autoclaved sterile slides with 10,000 cells/spot in a 25 μl volume/spot with the slide consisting of 2 spots/slide. Cells were allowed to attach and grown, enough to cover the slides and left for 24 hours at 37° C. Following day the media was changed and washed with PBS and 7 ml of serum free media was added for 24 hours at 37° C. After 24 hours, 1 ml of the various proteins were added; 2 nM and 8 μM human epidermal growth factor (hEGF) (10 and 50 ng/ml, respectively), TGFαPE40ΔAsp553 (70 and 350 ng/ml), and TGFαhingeCH2CH3 IgG (hIGD)(50 and 250 ng/ml) for 1-3 days. Slides were washed with PBS twice in coplin jars at room temperature not shaking. Cells were fixed in 4% paraformaldehyde solution in PBS, pH 7.4 for 25 minutes at 4° C. in coplin jars not shaking and then washed in PBS twice at 5 minutes each at room temperature. Slides were put into 0.2% Triton X-100 solution in PBS for 15 minutes at room temperature. The slides were rinsed in PBS. An excess of liquid was removed by tapping slides gently and each spot was covered with 100 μl of equilbration buffer for 10 minutes at room temperature while in a humidified chamber. The TdT incubation buffer solution was made according to the instruction manual (Promega). An excess of liquid was drained off and add 25 μl of the mixture was added per spot for 1 hour at 37° C. in the dark. 2×SSC solution in H₂O was add to slides for 15 minutes at room temperature in the dark. Then, the slides were washed in PBS 3 times 5 minutes each in the dark. An excess of liquid was drained off and cells were counterstained with Hoechst No. 33258 Nuclear Counterstain (DAPI) (1:1000) in 1.5% NGS/PBS for 15 minutes in the dark. The slides were washed in water 3 times at 5 minutes each in the dark. An excess of liquid was tapped off and each slide was mounted with GelMount (Biomeda Corp., Foster City, Calif., USA) and allowed to dry overnight at room temperature. Pictures were taken using a 40× magnification in all cases with a Hamamatsu C2400 digital camera. Background was normalized for each sample. All the images were taken at the same settings. Images were processed in Paint Shop Pro V 6.0 (Jasc Software Inc., Eden Prairie, Minn., USA).

In one set of experiments, the results showed that apoptosis was induced in G48a GBM cells by either EGF or TGFα-hIGD. Green immunofluorescence corresponds to fragmented DNA was observed in nuclei of apoptosis undergoing cells (Tunel assay). DAPI nuclear staining was analyzed for comparison. Cells were treated with either 2 or 8 nM of recombinant proteins. More green immunofluorescence was observed in the 8-nM treated cells than the 2 nM-treated cells. In another set of experiments, apoptosis was induced in GBM cells (G48a and U87), but not in glial cells, NIH3T3 cells, or HUVEC in response to treatment with 8 nM of EGF, TGFαPE40ΔAsp553, or TGFΔhingeCH2CH3.

### EXAMPLE 5

### Method of Testing EGFR Ligands for Ability to Induce Apoptosis in Cancer Cells In Vivo

The suitability of recombinant EGFR ligands for specific deliveries to brain tumors, such as high grade astrocytoma (HGA), can be determined in animal experiments. Tumors (U-87 MG, U-251 MG, and A431 as a positive control) are induced subcutaneously (s.c.) and intracranially (i.e.) in immunocompromised nu/nu mice. For s.c. tumors, 6×10⁶ tumor cells per mouse are inoculated in the right flank in a volume of 0.1 ml of excipient (ordinarily 5% methylcellulose in serum-free tissue culture medium). The tumors are allowed to grow to 200 to 250 mm³ as determined by calculation from length and width measurements obtained with digital vernier calipers. The formula for volume calculation is 0.4 ab² where a is the length and b is the greatest width perpendicular to the length.

The Maximum Tolerable Dose (MTD) is established in mice by 2 different routes: intravenous (i.v.) and intracerebral (i.e.). MTD is estimated as the dose that produces lethality in 10% of the mice (LD₁₀) within 21 days, depending on the administration route. A value that is 75% or less of the LD₁₀ value is used as the maximum dose in subsequent in vivo studies, although toxicity may not be observed even at high doses of recombinant EGFR ligands. Histopathologic examination of blood and various tissues taken from animals that become moribund from recombinant EGFR ligands toxicity are conducted to identify the target organ(s) and to establish/confirm the mechanism of toxicity.

The anti-tumor activity of recombinant EGFR ligands can also be evaluated in a glioma xenograft mouse model system. To quantify and compare efficacy of different recombinant EGFR ligands as anti-tumor agents, the survival of mice bearing syngeneic tumors is examined. Direct testing of efficacy is based on the capacity of single versus multiple injections of recombinant EGFR ligands to exert a demonstrable effect on tumor growth and progression in a mouse tumor model. Groups of mice implanted with glioma tumors (or A431) in the flanks are monitored with tumor measurements taken on a daily or every-other-day basis to reflect the anti-tumor effect of recombinant EGFR ligands administered by i.t. or i.v. routes. Survival groups consisting of 10-13 mice/group are followed until tumor volumes attain approximately 2,000 mm³ at which point they are sacrificed and necropsied. Relevant tissues are submitted for histopathologic examination. An investigation of the ability of recombinant EGFR ligands to affect the growth of gliomas implanted in the brains of nu/nu mice is evaluated. The delivery regimen begins with a single local injection of various recombinant EGFR ligands. Mice are treated 5 days after tumor induction and their median survival is the endpoint in these studies.

To examine the anti-tumor efficacy of recombinant EGFR ligands, survival analysis methods are employed. The vehicle and recombinant EGFR ligands Kaplan-Meier survival curves are compared via the log-rank test. The trend test, ordinally coding the vehicle and increasing doses of recombinant EGFR ligands, is employed in a proportional hazards model to assess the effect of recombinant EGFR ligands dosage on survival. Since many of the efficacy hypotheses are very exploratory in nature, the sample size calculation is lenient. To detect a hazard ratio of 3.25 as being significantly (statistically significant) different from 1.0, and assuming the probability of observing the event of interest (death) is 0.9, 13 mice per group are required for a two-sided α-level test having 80% power.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A chimeric molecule comprising an epidermal growth factor receptor ligand and a carrier molecule comprising a portion of an immunoglobulin molecule.

2. The chimeric molecule of claim 1, wherein the epidermal growth factor receptor ligand is TGFα.

3. The chimeric molecule of claim 1, wherein the portion of the immunoglobulin molecule comprises CH2 and CH3 domains.

4. The chimeric molecule of claim 1, wherein the portion of the immunoglobulin molecule further comprises a hinge region.

5. A chimeric molecule comprising an epidermal growth factor receptor ligand and a carrier molecule comprising PE40Δ553/Δ609-613.

6. The chimeric molecule of claim 5, wherein the epidermal growth factor receptor ligand is TGFα.

7. A nucleic acid encoding a chimeric molecule comprising an epidermal growth factor receptor ligand and a carrier molecule comprising a portion of an immunoglobulin molecule or PE40Δ553/Δ609-613.

8. A method of inducing apoptosis in a cancer cell, the method comprising the step of administering a composition comprising a chimeric molecule comprising an epidermal growth factor receptor ligand and a carrier molecule in an amount effective to induce apoptosis in the cell.

9. The method of claim 8, wherein the epidermal growth factor receptor ligand is TGFα.

10. The method of claim 8, wherein the carrier molecule comprises a portion of an immunoglobulin molecule.

11. The method of claim 10, wherein the portion of the immunoglobulin molecule comprises CH2 and CH3 domains.

12. The method of claim 11, wherein the portion of the immunoglobulin molecule further comprises a hinge region.

13. The method of claim 8, wherein the carrier molecule comprises PE40Δ553/Δ609-613.

14. The method of claim 9, wherein the carrier molecule comprises PE40Δ553/Δ609-613.

15. The method of claim 8, wherein the cancer cell is a glioma cell.

16. The method of claim 8, wherein the glioma cell is a glioblastoma multiforme cell.
